(19) United States
(12) Patent Application Publication
(10) Pub. No.: US 2008/0058410 A1
(43) Pub. Date: Mar. 6, 2008

(54) Composition for Stabilizing Vitamin C in Water Phase and Method for Stabilizing C Using Thereof

(75) Inventors: Chul-Hwan Kim, Daejeon (KR); Hyun-Nam Yoon, Towaco, NJ (US)

Correspondence Address: MARGER JOHNSON & MCCOLLOM, P.C., 210 SW MORRISON STREET, SUITE 400, PORTLAND, OR, 97204, US

(73) Assignee: DPI SOLUTIONS, INC., Daejeon 305-345 (KR)

(21) Appl. No.: 11/572,566

(22) PCT Filed: Jul. 23, 2004
(86) PCT No.: PCT/KR04/01840

§ 371 Date: Oct. 31, 2007

(51) Int. Cl. A61P 39/06; A61K 31/375; A61Q 99/00

(52) U.S. Cl. 514/474; 252/397; 252/400.1; 426/654

(57) ABSTRACT

The present invention relates to a composition for stabilizing vitamine C in water phase and method for stabilizing vitamine C using the composition. More particularly, this invention relates to a composition wherein the vitamine C is effectively prevented from decomposing by an exterior environment such as water, temperature, and light by stabilizing the vitamine C with the use of a cationic polymer and an anionic polymer, and thereof method for stabilizing vitamine C.

### TECHNICAL FIELD

The present invention relates to a composition for stabilizing vitamine C in water phase and method for stabilizing vitamine C using the composition. More particularly, this invention relates to a composition wherein the vitamine C is effectively prevented from decomposing by an exterior environment such as water, temperature, and light by stabilizing the vitamine C with the use of a cationic polymer and an anionic polymer, and thereof method for stabilizing vitamine C.

The ascorbic acid (vitamine C) improves the immuno function of human body, acceralates a production of a collagen, constituent of skin, cartilage, capillary, and muscle, and prevents from the damage of skin by decomposing the chemical substance generated by a ultraviolet rays that infiltrate into a skin. Also, the ascorbic acid prevents from a line or a wrinkle of skin, maintains healthily a skin, and assists a treatment of skin tissue damage. The ascorbic acid is known as an anti aging agent preventing from the formation of the histamine that may cause the allergy reaction and the melamine that may cause the faded skin during an aging.

However, the ascorbic acid, like γ-lactone, is unstable and easily reacts with the exterior environment such as air, oxygen, heat, and light. The oxidization reaction of the ascorbic acid produces the dihydroascorbate radical, the oxidization intermediate of the ascorbic acid due to the dissociation of the hydrogen ion, which is two sequential electron transfer process. The produced dihydroascorbate radical is very good for a reaction, and is known to generate one molecule of the ascorbic acid and one molecule of the dehydroascorbic acid. The minimum amount of the ascorbic acid dissolves in non-aqueous solution, but the large amount of the ascorbic acid dissolves in aqueous solution. However, only small amount of the ascorbic acid can be used as an active ingredient for medicine, food, and cosmetics because the sufficient amount of the ascorbic acid is not stabilized due to the fast oxidization.

In order to solve the inherent problems and improve the stability of the ascorbic acid, various prior methods of using the ascorbic acid derivative have been proposed, but they are bad for efficiency.

### BACKGROUND ART

Recently, there are proposed for preventing the oxidization of the pure ascorbic acid, methods of adding the antioxidant, approaches for stabilizing in multiple emulsion phase for preventing the oxidization of the ascorbic acid in formulation, methods for stabilizing the emulsion of the type of the oil in water (O/W), and methods for preventing the oxidization of the ascorbic acid using H₂SO₄ (Zinc Sulfate) and L-tyrosine (U.S. Pat. No. 4,938,969, EP 0533667 B1).

However, the proposed methods have the problems as following. The method of using the antioxidant is known to have some antioxidization effects when a phenol derivative such as a tocopherol is used as an antioxidant. However, the method has the defeats that brown color is generally changed into the violet color. Further, the antioxidant ability is weak in comparison with the antioxidant ability of the ascorbic acid, and therefore the method is not accepted for effective antioxidization of the ascorbic acid. As a method of using the antioxidant of mineral, use of the thiosulfate derivative has disadvantages in that the ascorbic acid, when dissolved, becomes the acidity of pH 2.0 to 4.0. And unpleasant smell is produced from SO₂ (Sulfur dioxide) freed by the equilibrium of the thiosulfates and the SO₂ (Sulfur dioxide) gas in the range from pH 2.0 to 4.0. In addition, the method of adding the various chemical synthetic antioxidants is incompatible with the conception that the pure ascorbic acid is employed and unsuitable for the practical application, and has problem that usage amount for stabilizing the ascorbic acid of high concentration is limited. On the one hand, the approach for stabilizing the ascorbic acid in multiple emulsion phase has shortcomings that even though the ascorbic acid is stabilized in multiple emulsion itself, the decrease of the titer is inevitable with the passage of time because most of multiple emulsion is inferior in stability over time. Also the method of stabilizing the ascorbic acid in oil phase is not suitable for use in the material of water phase such as a medicine and cosmetics because of difficulty in adding the ascorbic acid into the water material.

### DISCLOSURE OF THE INVENTION

Accordingly, the inventors repeated studies in order to solve the problems of conventional approaches, and finally perfect this invention that can prevent the ascorbic acid from being decomposed from the reaction with the exterior environment such as water, oxygen, heat, air, and light. In the present invention the ascorbic acid is capsulated by the chemicophysical combination with a polymer chain using a cationic polymer and an ionic polymer.

An object of the present invention is to provide a composition for stabilizing the ascorbic acid in water phase.

Another object of the present invention is to provide a method for stabilizing the ascorbic acid using the composition.

Another object of the present invention is to provide a pharmaceutical composition comprising the composition for stabilizing the ascorbic acid as an efficient ingredient.

Another object of the present invention is to provide a cosmetic composition comprising the composition for stabilizing the ascorbic acid as an efficient ingredient.

Another object of the present invention is to provide a food comprising the composition for stabilizing the ascorbic acid as an efficient ingredient.

### BEST MODES FOR CARRYING OUT THE INVENTION

In order to accomplish the objects, the composition according to the present invention comprises 5.0% to 25.0% by weight of an ascorbic acid, 0.1% to 5.0% by weight of a cationic polymer, 0.1% to 5.0% by weight of an anionic polymer, and 35.0% to 94.8% by weight of water.

Now, the present invention will be described in more detail.

The composition according to the present invention is based on the fact that the ascorbic acid becomes the anionic polymer by dissolving. The ascorbic acid in the composition is stabilized through the cationic polymer such as a chitosan and amino acid and ascorbic acid in the composition being combined to form stabilized acid-base complex (first stabilization); the complex being second capsulated by the anionic polymer such as a gelatine to make the ascorbic acid further stabilized. Therefore, the present invention can effectively prevent the ascorbic acid in water phase form being decomposed by the water and light.

The composition of the present invention may comprise the pure ascorbic acid as an ascorbic acid. However, the water may decompose the ascorbic acid in the composition before the ascorbic acid is stabilized, and thus the composition may comprise the form dissolved by a polyhydric alcohol. The polyhydric alcohol is preferable to be used by one or more selected from the consisting group of a propylene glycol, glycerine, 1,3-butandiol, and sorbitol. The usage amount of the polyhydric alcohol is preferable to 10.0% to 30.0% by weight based on the total weight of the composition, but the usage amount is not limited thereto.

The cationic polymer is not limited, but is preferable the polymer having more than two amine groups and harmless polymer in human body. The detailed examples of the cationic polymer include a chitosan, lysine, arginine, cystine, polyethyleneimine, and polyvinylpyrolidone. The complexing effect for the ascorbic acid is not generated when the cationic polymer of small amount is added into the composition. However, the problem of the eduction due to the solubility of the cationic polymer may occur when the cationic polymer of large amount is added into the composition. Therefore, the cationic polymer is preferable to be used in a range from 0.1% to 0.5% by weight based on the total weight of the composition.

Also, the composition according to the present invention may further comprise the anionic polymer in order to molecularly completely capsulate the ascorbic acid that forms the complex with the cationic polymer. The anionic polymer can completely separate the ascorbic acid from the water by dimensionally gathering around the ascorbic acid that forms the complex to combine with the cationic polymer. Furthermore, the anionic polymer may protect the ascorbic acid because the anionic polymer is able to absorb the ultraviolet rays in case of infiltrating the ultraviolet rays.

The kind of the anionic polymer in the composition according to the present invention is not limited. However, the anionic polymer is preferably a gelatine, hyaluronic acid, alginic acid, sodium alginic acid, starch, starch oxide, or carboxyl methylcellulose, and is more preferably the gelatine or hyaluronic acid. The intensity of the capsulation layer is improved while the solubility is limited, and the viscosity of the composition becomes high when the molecular weight of the gelatine or the hyaluronic acid is large. Therefore the gelatine or hyaluronic acid having large molecular weight is difficult to be used for the composition. It is preferable that the molecular weight of the gelatine ranges from about 100,000 to 1,000,000 and the molecular weight of the hyaluronic acid ranges from about 2,000,000 to 8,000,000.

The usage amount of the anionic polymer is preferable the same as the amount of the cationic polymer for the stabilization of the capsulation layer.

The composition according to the present invention may further comprise the antioxidant such as a tyrosine and tryptophan for further improving the effect for the stabilization of the ascorbic acid. The large amount of the antioxidant, which includes in structure both the amine group and carboxylic acid, decreases the stabilization effect for the ascorbic acid by blocking the amine group of the cationic polymer from forming the complex with the ascorbic acid due to the increase of the acidity of solution. Therefore, the antioxidant is preferable to be comprised less than 1.0% by weight based on the total weight of the composition.

The composition according to the present invention strengthens the combination of the ascorbic acid and the cationic polymer by neutralizing the hydrogen ion generated during the ionization of the ascorbic acid, and hence may further comprise the alkali additives in order to further improve the effect for stabilizing the ascorbic acid. The kind of the alkali additives is particularly not limited, but includes the sodium hydroxide, potassium hydroxide, calcium hydroxide, magnesium hydroxide, and aluminum hydroxide. It is preferable to choose the amount of the alkali additives in such a way that the composition has pH scale of less than pH 6.0.

The composition for stabilizing the ascorbic acid according to the present invention is suitable for use as active ingredients for cosmetics compositions, pharmaceutical material, and foods. For example, the composition can be variously applied to injections, and skin ointments in the form of the formulated jell, layer, bead, mesh, or coated fiber because the composition according to the present invention is harmless in human body. That is, the cell is activated, and the damage is fastly treated when the composition for stabilizing the ascorbic acid using chitosan is used as an injection. Moreover, the composition can provide the whitening effect or the removal effect of the homy substance when the composition is coated and dried on the nonwoven substrate of the gauze form. Further, when coated with the fiber the composition has gauze form and can be put on an injury to make quick recovery from injury.

The present invention can produce the cosmetics composition, pharmaceutical composition, and food comprising the composition for stabilizing the ascorbic acid as an active ingredient. The composition or the food can be produced as ordinary method in the field of the present invention. Specifically, the pharmaceutical composition and the food comprising the composition as an active ingredient can be produced following the step for adding the general vehicle pharmaceutically and food engineering allowed and the step for formulating the general form of medicine by the method of producing the pharmaceutical medicine and the method of food engineering.

The present invention provides an improved method for stabilizing the ascorbic acid in water phase using the composition, which comprises the step for dissolving the ascorbic acid in water, the step for adding the cationic polymer to the ascorbic acid aqueous solution, and the step for adding the anionic polymer to the solution mixed the ascorbic acid and the cationic polymer.

The ascorbic acid may be dissolved by the water before stabilized, therefore the ascorbic acid is preferable to be added to water after the ascorbic acid dissolves in the polyhydric alcohol to minimize the decomposition of the ascorbic acid in preparing the composition. The polyhydric alcohol is preferable to be selected more than one kind in the consisting group of the propylene glycol, glycerine, 1,3-butandiol and sorbitol. The usage amount is preferably used 10.0% to 30.0% by weight of based on the total weight of the composition, but is not limited thereto.

Further, each process in the present invention is preferable to be carried out in the condition of the temperature ranging from 10° C. to 25° C. The range of temperature is for minimizing the decomposition of the ascorbic acid by heat.

Next, the present invention is described in more detail using the examples and comparative examples, but is not limited thereto.

### EXAMPLES 1˜5 AND COMPARATIVE EXAMPLES 1˜2

TABLE 1

| | | | | | | | Comparative | |
|---|---|---|---|---|---|---|---|---|
| | | Examples | | | | | examples | |
| Composition | | 1 | 2 | 3 | 4 | 5 | 1 | 2 |
| (1) Glycerine | | — | — | 20.0 | 30.0 | 30.0 | — | 10.0 |
| (2) Water | | 83.4 | 83.4 | 57.55 | 47.6 | 47.6 | 85.0 | 83.2 |
| (3) | Lysine | — | — | — | 0.25 | 0.25 | — | 0.2 |
| Cationic | Argingine | 0.1 | 0.1 | — | — | — | — | — |
| Polymer | Cystine | — | — | 0.1 | — | — | — | — |
| | Chitosan | 0.75 | 0.75 | 0.75 | 0.75 | 0.75 | — | 0.5 |
| (4) | Gelatine | 0.75 | 0.75 | 1.0 | 0.75 | 0.75 | — | — |
| Anionic | Hyaluronic | — | — | 0.1 | 0.25 | 0.25 | — | — |
| Polymer | acid | | | | | | | |

| | | | | | | | |
|---|---|---|---|---|---|---|---|
| (5) Tryptophan | — | 0.1 | 0.5 | 0.4 | 0.4 | — | 0.1 |
| (6) Ascorbic acid | 15.0 | 15.0 | 20.0 | 20.0 | 20.0 | 15.0 | 5.0 |
| (7) Sodium Hydroxide | — | 5.0 | 10.0 | 15.0 | 15.0 | — | — |
| (0.5N) | | | | | | | |
| pH | 2.5 | 3.5 | 5.0 | 5.4 | 5.4 | 2.5 | 2.8 |

<Preparation Method>

Step (1): the water (2) or the mixture of water (2) and glycerine (1) was bottled in a beaker or a flask, and then the ascorbic acid dissolved.

Step (2): the cationic polymer (3) was added into the solution of step (1) at the room temperature and dissolved.

Step (3): the anionic polymer (4) was added into the solution of step (2) and dissolved, and the ascorbic acid (6) was capsulated by adding the tryptophan (5) and the sodium hydroxide (0.5N) (7).

### EXPERIMENTAL EXAMPLE 1

### Titer of the Ascorbic Acid

The primary titer of the ascorbic acid was set to 100 in the composition prepared by the examples 1˜5 and the comparative examples 1˜2. The titer of the ascorbic acid was measured at the room temperature, 37° C. and 45° C. respectively after a month, and the result was shown in Table 2. The titer was measured with remainder using the HPLC (was manufactured by Waters Company). In order to measure the remainder, the condition of HPLC was the detector wave of 254 nm and the flow rate of 0.8 ml/min using the Luna C18 column of Phenomenex Company. The standard measuring graph was drawn using the peak height showing in the 266 nm of the measured remainder and the ultraviolet spectroscope, and the amount of the ascorbic acid was relatively determined using the ultraviolet spectroscope. The ultraviolet spectroscope was used He λ ios β kind of Spectronic Unicam Company.

TABLE 2

| | | | | | | Comparative | |
|---|---|---|---|---|---|---|---|
| | Example | | | | | example | |
| | 1 | 2 | 3 | 4 | 5 | 1 | 2 |
| Room Temperature | 90 | 94 | 97 | 99 | 99 | 40 | 75 |
| 37° C. | 85 | 90 | 95 | 98 | 98 | 31 | 65 |
| 45° C. | 79 | 88 | 94 | 97 | 97 | 15 | 57 |

As shown in the table 2, the ascorbic acid was capsulated with the cationic polymer and the anionic polymer in the examples 1˜5. The decrease of titer for ascorbic acid in the examples 1˜5 was smaller than the comparative example 1 comprising only ascorbic acid and than comparative example 2 stabilizing with the cationic polymer in accordance with the passage of time.

### INDUSTRIAL APPLICABILITY

As above described, the composition according to the present invention is to improves the stability by capsulating ascorbic acid. The composition can be valuably used for the cosmetics and the medical supplies, and is excellent molecular assembly form having the value in use due to the water phase. Particularly, the ascorbic acid can be prevented from decomposing by the exterior environment such as water, temperature, and light by stabilizing the ascorbic acid with the novel method.

What is claimed is:

1. A composition for stabilizing ascorbic acid in water phase comprising:
5.0% to 25.0% by weight of an ascorbic acid;
0.1% to 5.0% by weight of a cationic polymer;
0.1% to 5.0% by weight of an anionic polymer, and
35.0% to 94.8% by weight of water.

2. The composition of claim 1 further comprising less than 1.0% by weight of an antioxidant based on the total weight of the composition.

3. The composition of claim 1 further comprising a sodium hydroxide, potassium hydroxide, calcium hydroxide, magnesium hydroxide, and aluminum hydroxide, and wherein a pH scale is controlled to be within less than pH 6.0.

4. The composition of claim 1 to claim 3, wherein the ascorbic acid is dissolved by more than a kind of polyhydric alcohol selected from the consisting group of a propylene glycol, glycerine, 1,3-butandiol, and sorbitol.

5. The composition of claim 1 to claim 3, wherein the cationic polymer is a chitosan, lysine, arginine, cystine, polyethyleneimine, or polyvinylpyrrolidone.

6. The composition of claim 1 to claim 3, wherein the anionic polymer is a gelatine, hyaluronic acid, alginic acid, sodium alginic acid, starch, starch oxide, or carboxymethylcellulose.

7. The composition of claim 1, which is formulated with form of a gel, layer, bead, mesh, or coated fiber.

8. A method of stabilizing an ascorbic acid in water phase comprising:
(1) dissolving an ascorbic acid in water,
(2) adding a cationic polymer to the ascorbic acid aqueous solution of step (1) and
(3) adding an anionic polymer to the solution mixed the ascorbic acid and the cationic polymer of step (2).

9. The method of claim 8, wherein the ascorbic acid is dissolved by more than a kind of polyhydric alcohol selected from the consisting group of a propylene glycol, glycerine, 1,3-butandiol, and sorbitol.

10. The method of claim 8, wherein the cationic polymer is a chitosan, lysine, arginine, cystine, polyethyleneimine, or polyvinylpyrrolidone.

11. The method of claim 8, wherein the anionic polymer is a gelatin, hyalurionic acid, alginic acid, sodium, alginic acid, starch, starch oxide, or carboxymethylcellulose.

12. The method of claim 8, wherein the each process is performed at the temperature ranging from 10° C. to 25° C.

13. A cosmetic composition comprising composition for stabilizing the ascorbic acid of claim 1 as an active ingredient.

14. A pharmaceutical composition comprising composition for stabilizing the ascorbic acid of claim 1 as an active ingredient.

15. A food comprising composition for stabilizing the ascorbic acid of claim 1 as an active ingredient.
